(19) United States
(12) Patent Application Publication
(10) Pub. No.: US 2021/0259192 A1
(43) Pub. Date: Aug. 26, 2021

(54) UNIQUE HIGH YIELD SOYBEAN CULTIVARS

(71) Applicant: FTE GENETICS, INC., OSKALOOSA, IA (US)

(72) Inventors: Kyle Olson, Oskaloosa, IA (US); Gerald Lorenzen, Oskaloosa, IA (US)

(21) Appl. No.: 17/256,752

(22) PCT Filed: Jul. 5, 2019
(86) PCT No.: PCT/US19/40732

§ 371 Date: Dec. 29, 2020

Related U.S. Application Data
(60) Provisional application No. 62/694,738, filed on Jul. 6, 2018.

(51) Int. Cl. A01H 6/54; A01H 5/10

(52) U.S. Cl. CPC A01H 6/542; A01H 5/10

(57) ABSTRACT

According to the instant one or more embodiments, there is provided new soybean cultivars designated FTE 2009, FTE 3049, and FTE 3140. The one or more embodiments thus relate to the seeds of soybean cultivars FTE 2009, FTE 3049, and FTE 3140, to the plants of soybean cultivars FTE 2009, FTE 3049, and FTE 3140, to plant parts of soybean cultivars FTE 2009, FTE 3049, and FTE 3140, and to processes for producing a soybean plant produced by crossing soybean cultivars FTE 2009, FTE 3049, and FTE 3140 with itself or with another soybean cultivar, and the creation of variants by mutagenesis or transformation of soybean cultivars FTE 2009, FTE 3049, and FTE 3140. The one or more embodiments also relate to the commercial commodity products from the seeds of soybean cultivars FTE 2009, FTE 3049, and FTE 3140, and the food products comprising these commercial commodity products.

### CROSS-REFERENCE TO RELATED APPLICATIONS

The application is a 371 application of International Application No. PCT/US19/40732, filed Jul. 5, 2019, pending, entitled “UNIQUE HIGH YIELD SOYBEAN CULTIVARS”, which claims benefit from U.S. Provisional Application No. 62/694,738, filed Jul. 6, 2018, entitled “UNIQUE HIGH YIELD SOYBEAN CULTIVARS,” now expired. Each application listed above is hereby incorporated by reference in its entirety as if fully restated herein.

### BACKGROUND

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possesses the traits to meet the program goals. The goal is to combine in a single cultivar an improved combination of desirable traits from the parental germplasm.

Choice of breeding or selection process depends on the mode of plant reproduction, the heritability of the trait(s) being improved, and the type of cultivar used commercially (e.g., F₁ hybrid cultivar, pure line cultivar, etc.). For highly heritable traits, a choice of superior individual plants evaluated at a single location will be effective, whereas for traits with low heritability, selection should be based on mean values obtained from replicated evaluations of families of related plants. Popular selection process commonly include pedigree selection, modified pedigree selection, mass selection, and recurrent selection.

The complexity of inheritance influences choice of the breeding process. Backcross breeding is used to transfer one or a few favorable genes for a highly heritable trait into a desirable cultivar. This approach comprises been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested and compared to appropriate standards in environments representative of the commercial target area(s) for three or more years. The best lines are candidates for new commercial cultivars; those still deficient in a few traits may be used as parents to produce new populations for further selection.

These processes, which lead to the final step of marketing and distribution, usually take from 4 to 12 years from the time the first cross is made. Therefore, development of new cultivars is a time-consuming process involving precise forward planning, efficient use of resources, and a minimum of changes in direction.

A most difficult task is the identification of individuals that are genetically superior, because for most traits the true genotypic value is masked by other confounding plant traits or environmental factors. One process of identifying a superior plant is to observe its performance relative to other experimental plants and to a widely grown standard cultivar. These observations are usually replicated to ensure adequate data to estimate genetic worth sufficiently.

When attempting to use soybean plant breeding to develop new, unique and superior soybean cultivars and hybrids, the breeder initially selects and crosses two or more parental lines, followed by repeated selfing and selection, producing many new genetic combinations. The breeder can theoretically generate billions of different genetic combinations via crossing, selfing and mutations. The breeder comprises no direct control at the cellular level. Therefore, two breeders typically do not develop the same line, or even very similar lines, having the same soybean traits.

Each year, when developing a cultivar, the plant breeder selects the germplasm to advance to the next generation. This germplasm is grown under unique and different geographical, climatic and soil conditions, and further selections are then made, during and at the end of the growing season. The cultivars which are developed are unpredictable. This unpredictability is because the breeder's selection occurs in unique environments, with no control at the DNA level (using conventional breeding procedures), and with millions of different possible genetic combinations being generated.

A breeder of ordinary skill in the art cannot predict the final resulting lines he develops. This unpredictability results in the expenditure of large amounts of research monies to develop superior new soybean cultivars.

The development of new soybean cultivars involves the development and selection of soybean varieties, the crossing of these varieties, and the selection of superior hybrid crosses. The hybrid seed is produced by manual crosses between selected male-fertile parents or by using male sterility systems. These hybrids are selected for certain single gene traits such as pod color, flower color, pubescence color, plant physical characteristics, or disease resistance which indicate that the seed is truly a hybrid. Additional data on parental lines, as well as the phenotype of the hybrid, influence the breeder's decision whether to continue with the specific hybrid cross.

Pedigree breeding and recurrent selection breeding processes are used to develop cultivars from breeding populations. Breeding programs combine desirable traits from two or more cultivars or various broad-based sources into breeding pools from which cultivars are developed by selfing and selection of desired phenotypes. The new cultivars are evaluated to determine which comprise commercial potential.

Pedigree breeding is used commonly for the improvement of self-pollinating crops. Two parents which possess favorable, complementary traits are crossed to produce an F₁. An F₂ population is produced by selfing one or several F₁s. Selection of the best individuals may begin in the F₂ population; then, beginning in the F₃, the best individuals in the best families are selected. Replicated testing of families can begin in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable homozygous cultivar or inbred line which is the recurrent parent. The source of the trait to be transferred is called the donor parent. The resulting plant is expected to comprise the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to comprise the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In a multiple-seed procedure, soybean breeders commonly harvest one or more pods from each plant in a population and thresh them together to form a bulk. Part of the bulk is used to plant the next generation and part is put in reserve. The procedure has been referred to as modified single-seed descent or the pod-bulk technique.

The multiple-seed procedure has been used to save labor at harvest. It is considerably faster to thresh pods with a machine than to remove one seed from each by hand for the single-seed procedure. The multiple-seed procedure also makes it possible to plant the same number of seeds of a population each generation of inbreeding. Enough seeds are harvested to make up for those plants that did not germinate or produce seed.

Descriptions of other breeding processes that are commonly used for different traits and crops can be found in one of several reference books (e.g., Allard, 1960; Simmonds, 1979; Sneep et al., 1979; Fehr, 1987).

Proper testing should detect any major faults and establish the level of superiority or improvement over current cultivars. In addition to showing superior performance, there should be a demand for a new cultivar that is compatible with industry standards or which creates a new market. The introduction of a new cultivar will incur additional costs to the seed producer, the grower, processor and consumer; for special advertising and marketing, altered seed and commercial production practices, and new product utilization. The testing preceding release of a new cultivar should take into consideration research and development costs as well as technical superiority of the final cultivar. For seed-propagated cultivars, it should be feasible to produce seed easily and economically.

Nonetheless, a need exists for one or more alternative, situationally-improved, or high yield soybean cultivars.

### SUMMARY

The disclosure below uses different prophetic embodiments to teach the broader principles with respect to articles of manufacture, apparatuses, processes for using the articles and apparatuses, processes for making the articles and apparatuses, and products produced by the process of making, along with necessary intermediates. This Summary is provided to introduce ideas herein that a selection of concepts is presented in a simplified form as further described below. This Summary is not intended to identify key features or essential features of subject matter, nor is this Summary intended to be used to limit the scope of claimed subject matter. Additional aspects, features, and/or advantages of examples will be indicated in part in the description which follows and, in part, will be apparent from the description, or may be learned by practice of the disclosure.

Without intent to limit the scope of the disclosure, examples of methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions will control.

With the foregoing in mind, consider as one or more embodiments any of three new soybean cultivars designated FTE 2009, FTE 3049, and FTE 3140. The one or more embodiments of the disclosure thus relate to the seeds of soybean cultivars FTE 2009, FTE 3049, and FTE 3140, to the plants of soybean cultivars FTE 2009, FTE 3049, and FTE 3140, to plant parts of soybean cultivars FTE 2009, FTE 3049, and FTE 3140, and to process for producing a soybean plant produced by crossing soybean cultivars FTE 2009, FTE 3049, and FTE 3140 with itself or with another soybean cultivar, and the creation of variants by mutagenesis or transformation of soybean cultivars FTE 2009, FTE 3049, and FTE 3140. The one or more embodiments of the disclosure also relate to the commercial commodity products from the seeds of soybean cultivars FTE 2009, FTE 3049, and FTE 3140, and the food products comprising these commercial commodity products.

Thus, any such process using the soybean cultivars FTE 2009, FTE 3049, and FTE 3140 are part of one or more embodiments of the disclosure: selfing, backcrosses, hybrid production, crosses to populations, and the like. All plants produced using soybean cultivars FTE 2009, FTE 3049, and FTE 3140 as at least one parent are within the scope of one or more embodiments of the disclosure. These soybean cultivars can be used in crosses with other, different, soybean plants to produce first generation (F₁) soybean hybrid seeds and plants with superior characteristics.

In another aspect, the present one or more embodiments of the disclosure provides for single or multiple gene converted plants of soybean cultivars FTE 2009, FTE 3049, and FTE 3140. The transferred gene(s) may be a dominant or recessive allele. The transferred gene(s) may confer such traits as herbicide resistance, insect resistance, resistance for bacterial, fungal, or viral disease, male fertility, male sterility, enhanced nutritional quality (e.g., increased protein content), decreased seed size, size shape, yield, germination ability, and industrial usage. The gene may be a naturally occurring soybean gene or a transgene introduced through genetic engineering techniques.

In another aspect, the present one or more embodiments of the disclosure provides regenerable cells for tissue culture of soybean plants FTE 2009, FTE 3049, and FTE 3140. The tissue culture will in most cases be capable of regenerating plants having the physiological and morphological characteristic of the foregoing soybean plant, and of regenerating plants having substantially the same genotype as the foregoing soybean plant. The regenerable cells in such tissue cultures may be embryos, protoplasts, meristematic cells, callus, pollen, leaves, anthers, roots, root tips, flowers, seeds, pods or stems. Still further, the present one or more embodiments of the disclosure provides soybean plants regenerated from the tissue cultures of one or more embodiments of the disclosure.

The one or more embodiments of the disclosure also relate to process for producing a soybean plant containing in its genetic material one or more transgenes and to the transgenic soybean plants and plant parts produced by those process. One or more embodiments of the disclosure also relate to soybean cultivars or breeding cultivars and plant parts derived from soybean cultivars FTE 2009, FTE 3049, and FTE 3140, to process for producing other soybean cultivars, lines or plant parts derived from soybean cultivars FTE 2009, FTE 3049, and FTE 3140 and to the soybean plants, varieties, and their parts derived from use of those process, including traditional breeding and genetic engineering. The one or more embodiments of the disclosure further relate to hybrid soybean seeds, plants and plant parts produced by crossing soybean cultivars FTE 2009, FTE 3049, and FTE 3140 with another soybean cultivar.

### DETAILED DESCRIPTION

### Definitions

In the description and tables which follow, a number of terms are used. In order to provide a clear and consistent understanding of the specification and claims, including the scope to be given such terms, the following definitions are provided:

Allele: Allele is any or one of more alternative forms of a gene, all of which relate to one trait or characteristic. In a diploid cell or organism, the two alleles of a given gene occupy corresponding loci on a pair of homologous chromosomes.

Backcrossing: Backcrossing is a process in which a breeder repeatedly crosses hybrid progeny back to one of the parents, for example, a first-generation hybrid F₁ with one of the parental genotypes of the F₁ hybrid.

Cotyledon: A cotyledon is a type of seed leaf. The cotyledon contains the food storage tissues of the seed.

Disease Resistance: Disease resistance genes comprise the ability to detect a pathogen attack and facilitate a counter attack again the pathogen.

Embryo. The embryo is the small plant contained within a mature seed.

Emergence: Emergence is the score that indicates the ability of the seed to emerge when planted 3″ deep in sand and with a controlled temperature of 25 C. The number of plants that emerge each day are counted. Based on this data, each genotype is given a 1 to 9 score based on its rate of emergence and percent of emergence. A score of 9 indicates an excellent rate and percent of emergence, and intermediate score of 5 indicates average ratings and a 1 score indicates a very poor rate and percentage of emergence.

Hilum: Hilum refers to the scar left on the seed which marks the place where the seed was attached to the pod prior to the seed being harvested.

Hypocotyl: A hypocotyl is the portion of an embryo or seedling between the cotyledons and the root. Therefore, it can be considered a transition zone between shoot and root.

Maturity Group: Maturity Group refers to an agreed-on industry division of groups of plant varieties, based on Zones in which they are adapted primarily according to day length or latitude. They consist of very long day length varieties (Groups 000, 00, 0), to very short day length varieties (Groups VII, VIII, X). Group I includes the day length or latitude that includes Minnesota, South Dakota, and Nebraska. Subgroups refers to agreed-on industry division of Zones into parts of Zones.

Plant height: Plant height is taken from the top of the soil to the top node of the plant and is measured in centimeters.

Pod: Pod refers to the fruit of a soybean plant. It includes of the hull or shell (pericarp) and the soybean seeds.

Protein Percent: Soybean seeds contain a considerable amount of protein. Protein is generally measured by NIR spectrophotometry and is reported on an as is percentage basis.

Pubescence. This refers to a covering of very fine hairs closely arranged on the leaves, stems and pods of the soybean plant.

Quantitative Trait Loci (QTL): QTL refer to genetic loci that control to some degree numerically representable traits that are usually continuously distributed.

Regeneration: Regeneration refers to the development of a plant from tissue culture.

Shattering Resistance: Shatter resistance is the tendency of soybean pods to remain closed (i.e., sealed) and intact during and after maturity. The seal that keeps the soybean pod closed is intact and strong.

Seed Protein Peroxidase Activity: Seed protein peroxidase activity refers to a chemical taxonomic technique to separate cultivars based on the presence or absence of the peroxidase enzyme in the seed coat. There are two types of soybean cultivars: Those having high peroxidase activity (dark red color) and those having low peroxidase activity (no color).

Seed Yield (Bushels/Acre). The yield in bushels/acre is the yield of the grain at harvest.

Seeds Per Pound: Soybean sees vary in seed size, therefore, the number of seeds required to make up one pound also varies. This affects the pounds of seed required to plant a given area and can also impact end uses. Usually soybeans of the current one or more embodiments of the disclosure were measured as weight per 100 seeds.

Shattering. The amount of pod dehiscence prior to harvest. Pod dehiscence involves seeds falling from the pods to the soil. This is a visual score from 1 to 5 comparing all genotypes within a given test. A score of 1 means pods have not opened and no seeds have fallen out (i.e., no shattering, no dehiscence). A score of 5 indicates that 100% of the pods have opened.

Single Gene Converted (Conversion): Single gene converted (conversion) plant refers to plants which are developed by a plant breeding technique called backcrossing wherein essentially all of the desired morphological and physiological characteristic of a cultivar are removed in addition to the single gene transferred into the cultivar via the backcrossing technique or via genetic engineering.

Stem Vine Length: Stem vine length is measure of the stem in centimeters from the apical meristem of the plant to where the stem meets the ground and is recorded when plants are mature (that is, after flowering when pods are fully swollen).

Soybean cultivar FTE 2009 is a maturity group I soybean cultivar. FTE 2009 comprises very high yield potential and protein content when compared to lines of similar maturity and comprises semi-tolerance/resistance for several plant diseases including iron deficiency chlorosis, soybean cyst nematode, Phytophthora root rot, white mold, and soybean sudden death syndrome. Soybean cultivar FTE 3049 is a maturity group I, subgroup 6 cultivar. FTE 3049 comprises very high yield potential and protein content when compared to lines of similar maturity and comprises semi-tolerance/resistance for several plant diseases including iron deficiency chlorosis, soybean cyst nematode, Phytophthora root rot, white mold, and soybean sudden death syndrome. Soybean cultivar FTE 3140 is a maturity group I, subgroup 7 cultivar. FTE 3140 comprises very high yield potential and protein content when compared to lines of similar maturity and comprises semi-tolerance/resistance for several plant diseases including iron deficiency chlorosis, soybean cyst nematode, Phytophthora root rot, white mold, and soybean sudden death syndrome. These soybean cultivars have an aggressive root system that allows for quicker emergence and higher yield in lesser quality soils and different maturity group conditions.

Some of the criteria used to select in various generations include: seed yield, lodging resistance, emergence, disease tolerance, maturity, plant height, shattering resistance, and protein content.

References sited herein are incorporated by reference as if fully stated herein. The following description is illustrative and is not to be construed as limiting. Numerous specific details are described to provide a thorough understanding of the disclosure. However, in certain instances, well-known or conventional details are not described in order to avoid obscuring the description. References to one or an embodiment in the present disclosure can be, but not necessarily are, references to the same embodiment; and, such references mean at least one of the embodiments.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way.

Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

These cultivars have shown uniformity and stability, as described in the following cultivar description information. They have been self-pollinated a sufficient number of generations with careful attention to uniformity of plant type. These cultivars have been increased with continued observations for uniformity.

TABLE 1
Cultivar Characteristics

| | FTE 2009 | FTE 3049 | FTE 3140 |
|---|---|---|---|
| Location | Willmar, MN | Cannon Falls, MN | Cannon Falls, MN |
| tested | | | |
| Seed Shape | Round | Semi Round | Semi Round |
| Seed Coat | Yellow/ | Yellow/ | Yellow/ |
| Color | White | White | White |
| Seed Coat | Dull | Dull | Dull |
| Luster | | | |
| Seed Size | 20.3 g/ | 19.9 g/ | 19.5 g/ |
| | 100 Seeds | 100 Seeds | 100 Seeds |
| Hilum Color | Clear | Clear | Clear |
| Leaf Color | Green | Green | Green |
| Flower Color | White | White | White |
| Pod Color | Brown | Brown | Brown |
| Pubescence | Brown | Brown | Brown |
| Color | | | |
| Plant Type | Bush | Bush | Bush |
| Plant Height | Medium | Tall | Tall |
| Plant Growth | Determinate | Semi | Semi |
| Type | | Determinate | Determinate |
| Plant Habit | Erect | Erect | Erect |
| Maturity | I | I | I |
| Group | | | |
| Maturity | 0 | 6 | 7 |
| Subgroup | | | |
| Herbicide | Susceptible to | Susceptible to | Susceptible to |
| Reaction | Glyphosate | Glyphosate | Glyphosate |
| Iron | Semi | Semi | Semi |
| Chlorosis | Tolerant | Tolerant | Tolerant |
| Rating | | | |
| Cyst | Moderately | Resistant | Resistant |
| Nematode | Resistant | | |
| Rating | | | |
| Phytophthora | Semi | Semi | Semi |
| Root | Tolerant | Tolerant | Tolerant |
| Rot | | | |
| White Mold | Tolerant | Tolerant | Tolerant |
| Sudden Death | Tolerant | Semi | Semi |
| Syndrome | | Tolerant | Tolerant |
| Brown Stem | Semi | Tolerant | Tolerant |
| Rot | Tolerant | | |

Table 1 includes the morphologic and other characteristics of soybean cultivars FTE 2009, FTE 3049, and FTE 3140 based on data collected in Cannon Falls, Minn. or Willmar, Minn., which are maturity group I.

This disclosure is also directed to process for producing a soybean plant by crossing a first parent soybean plant with a second parent soybean plant, wherein the first or second soybean plant is the soybean plant from FTE 2009, FTE 3049, or FTE 3140. Further, both first and second parent soybean plants may be FTE 2009, FTE 3049, or FTE 3140. Therefore, any process using FTE 2009, FTE 3049, or FTE 3140 are part of the disclosure: selfing, backcrosses, hybrid breeding, and crosses to populations, and the like Any plants produced using FTE 2009, FTE 3049, or FTE 3140 as a parent are within the scope of this disclosure.

Useful processes include but are not limited to expression vectors introduced into plant tissues using a direct gene transfer process such as microprojectile-mediated delivery, DNA injection, electroporation and the like. More so expression vectors are introduced into plant tissues by using either microprojectile-mediated delivery with a biolistic device, of by using Agrobacterium-mediated transformation. Transformant plants obtained in the protoplasm of the one or more embodiments of the disclosure are intended to be within the scope of one or more embodiments of the disclosure.

### Further Embodiments of the Disclosure

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Such foreign additional and/or modified genes are referred to herein collectively as “transgenes”. Over the last twenty years several process for producing transgenic plants have been developed and the present one or more embodiments of the disclosure, in particular embodiments, also relates to transformed versions of the claimed cultivar or line.

Plant transformation involves the construction of an expression vector which will function in plant cells. Such a vector comprises DNA comprising a gene under control of, or operatively linked to, a regulatory element (for example, a promoter). The expression vector(s) may be in the form of a plastid and can be used alone or in combination with other plasmids to provided transformed soybean plants using transformation process to incorporate transgenes into the genetic material of the soybean plants(s).

Expression vectors include at least one genetic marker operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection process are also known in the art.

Genes included in expression vectors are driven by nucleotide sequence comprising a regulatory element, for example, a promoter. Several types of promoters are now well known in the transformation arts, as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids or sclerenchyma.

An inducible promoter is operably linked to a gene for expression in soybean. Optionally, the inducible promoter is operably linked to a nucleotide sequence encoding a signal sequence which is operably linked to a gene for expression in soybeans. An inducible promoter can be used in the instant one or more embodiments of the disclosure.

A constitutive promoter is operably linked to a gene for expression in soybean or the constitutive promoter is operably linked to a nucleotide sequence encoding a signal sequence with is operably linked to a gene for expression in soybean. Many different constructive promoters can be utilized in the instant one or more embodiments of the disclosure.

A tissue-specific promoter is operably linked to a gene for expression in soybean. Optionally, the tissue-specific promoter is operably linked to a nucleotide sequence encoding a signal sequence which is operably linked to a gene for expression in soybean. Plants transformed with a gene of interest operably linked to a tissue-specific promoter product the protein product of the transgene exclusively, or preferentially, in a specific tissue. Any tissue-specific or tissue-preferred promoter can be utilized in the instant one or more embodiments of the disclosure.

With transgenic plants according to the present one or more embodiments of the disclosure, a foreign protein can be produced in commercial quantities. Thus, techniques for the selection and propagation of transformed plants, which are well understood in the art, yield a plurality of transgenic plants which are harvested in conventional manner and a foreign protein then can be extracted from a tissue of interest or from total biomass. Protein extraction from plant biomass can be accomplished by known process.

According to an embodiment, the transgenic plant provided for commercial production of foreign protein is a soybean plant. In another embodiment, the biomass of interest is seed. For the relatively small number of transgenic plants that show higher levels of expression, a genetic map can be generated, primarily via conventional RFLP, PCR and SR analysis, which identified the approximate chromosomal location of the integrated DNA molecule. For exemplary methodologies in this regard, see Glick and Thompson, Methodsin Plant Molecular Biology and Biotechnology, (CRC Press, Boca Raton) 269:284 (1993). Map information concerning chromosomal locations is useful for proprietary protection of a subject transgenic plant. If unauthorized propagation is undertaken and crosses made with other germplasm, the map of the integration region can be compared to similar maps for suspect plants, to determine if the latter comprise a common parentage with the subject plant. Map comparisons can involve hybridizations, RFLP, PCR, SSR and sequencing.

Likewise, by means of the present one or more embodiments of the disclosure, agronomic genes can be expressed in transformed plants. More particularly, plants can be genetically engineered to express various phenotypes of agronomic interest. Exemplary genes implicated in this regard include but are not limited to, those categorized as genes that confer resistance to pests or disease, genes that confer resistance to an herbicide, and genes that confer or contribute to a value-added trait, including but not limited to higher protein content, higher oil content, seed roundness, and larger root system,

As to genes that confer resistance to pests or disease: plant defenses are often activated by specific interaction between the product of a disease resistance gene (R) in the plant and the product of a corresponding avirulence (Ar) gene in the pathogen. A plant cultivar can be transformed with one or more cloned resistance genes to engineer plants that are resistant to specific pathogen strains. Engineered plants that contain these genes are intended to be within the scope of one or more embodiments of the disclosure.

As to genes that confer resistance to an herbicide, an herbicide that inhibits the growing point or meristem, such as an imidazalinone or a sulfonylurea, or an herbicide that inhibits photosynthesis. A plant cultivar can be transformed with one of more of cloned resistance genes to engineered plants that are resistant to specific herbicides. Engineered plants that contain these genes are intended to be within the scope of one or more embodiments of the disclosure.

As to genes that confer or contribute to a value-added trait, such as modified fatty acid metabolism to increase plant stearic acid content, and such as modified carbohydrate composition effected, and increased protein content can be included in a transformation of a plant cultivar. Engineered plants that contain these genes that code (or confer) a value-added trait are intended to be within the scope of one or more embodiments of the disclosure.

Numerous processes for plant transformation have been developed, including biological and physical plant transformation protocols. See, for example, Mild et al., “Procedures for Introducing Foreign DNA into Plants” in Methods in Plant Molecular Biology and Biotechnology, Glick B. R. and Thompson, J. E. Eds. (CRC Press, Inc., Boca Raton, 1993) pages 67-88. In addition, expression vectors and in vitro culture process for plant cell or tissue transformation and regeneration of plants are available. See, for example, Gruber et al., “Vectors for Plant Transformation” in Methods in Plant Molecular Biology and Biotechnology, Glick B. R. and Thompson, J. E. Eds. (CRC Press, Inc., Boca Raton, 1993) pages 88-119.

Process for Soybean Transformation:

A. Agrobacterium-mediated Transformation: One process for introducing an expression vector into plants based on the natural transformational system of Agrobacterium. See, for example, Moloney et al., Plant Cell Reports 8: 238 (1989).

B. Direct Gene Transfer: Several processes of plant transformation, collectively referred to as direct gene transfer, have been developed as an alternative to Agrobacterium-mediated transformation. A generally applicable process of plant transformation is microprojectile-mediated transformation wherein DNA is carried on the surface of microprojectiles measuring 1 to 4 um. The expression vector is introduced into plant tissues with a biolistic device that accelerates the microprojectiles to sufficient speed to penetrate plant cell walls and membranes. Another process for physical delivery of DNA to plants is sonication of target cells. Direct uptake of DNA into protoplasts using CaCl₂ precipitation, polyvinyl alcohol or poly-L-orthinine comprises also been reported.

Following transformation of soybean target tissues, expression of the above-described selectable marker genes allows for preferential selection of transformed cells, tissues, and/or plants, using regeneration and selection process now well known in the art.

The foregoing process for transformation would typically be used for producing a transgenic cultivar. The transgenic cultivar could then be crossed with another (non-transformed or transformed) cultivar, in order to produce a new transgenic cultivar. Alternatively, a genetic trait which has been engineered into a particular soybean line using the foregoing transformation techniques could be moved into any of the line using traditional backcrossing techniques that are well known in the plant breeding arts. For example, a backcrossing approach could be used to move an engineered trait from a public, non-elite cultivar into an elite cultivar, or from a cultivar containing a foreign gene in its genome into a cultivar or cultivars which do not contain that gene. As used herein, “crossing” can refer to a simple X by Y cross, or the process of backcrossing depending on the context.

Single Gene Conversions of Soybean:

When the term soybean plant is used in the context of the present one or more embodiments of the disclosure, this also includes any single gene conversions of that cultivar. The term single gene converted plant as use herein refers to those soybean plants which are developed by a plant breeding technique called backcrossing wherein essentially all of the desired morphological and physiological characteristics of a cultivar are recovered in addition to the single gene transferred into the cultivar via the backcrossing technique. Backcrossing process can be used with the present one or more embodiments of the disclosure to improve or introduce a characteristic into the cultivar. The term backcrossing as used herein refers to the repeated crossing of a hybrid progeny back to the recurrent parent, i.e., crossing back 1, 2, 3, 4, 5, 6, 7, 8, 9, or more times to the recurrent parent. The parental soybean plant which contributes the gene for the desired characteristic is termed the nonrecurrent or donor parent. This terminology refers to the fact that the nonrecurrent parent is used one time in the backcross protocol and therefore does not recur. The parental soybean plant to which the gene or genes from the nonrecurrent parent are transferred is known as the recurrent parent as it is used from several rounds in the backcrossing protocol. In a typical backcross protocol, the original cultivar of interest (recurrent parent) is crossed to a second cultivar (nonrecurrent parent) that carries the single gene of interest to be transferred. The resulting progeny from this cross are then crossed again to the recurrent parent and the process is repeated until a soybean plant is obtained wherein essentially all of the desired morphological and physiological characteristics of the recurrent parent are recovered in the converted plant, in addition to the single transferred gene from the nonrecurrent parent as determined at the 5% significance level when grown in the same environmental conditions.

The selection of a suitable recurrent parent is an important step for a successful backcrossing procedure. The goal of a backcross protocol is to alter or substitute a single trait or characteristic in the original cultivar. To accomplish this, a single gene of the recurrent cultivar is modified or substituted with the desired gene from the nonrecurrent parent, while retaining essentially all of the rest of the desired genetic, and therefore the desired physiological and morphological, constitution of the original cultivar. The choice of the particular nonrecurrent parent will depend on the purpose of the backcross; one of the major purposes is to add some commercially desirable, agronomically important trait to the plant. The exact backcrossing protocol will depend on the characteristic or trait being altered to determine an appropriate testing protocol. Although backcrossing processes are simplified when the characteristic being transferred is a dominant allele, a recessive allele may also be transferred. In this instance it may be necessary to introduce a test of the progeny to determine if the desired characteristic has been successfully transferred.

Many single gene traits have been identified that are not regularly selected for in the development of a new cultivar but that can be improved by backcrossing techniques. Single gene traits may or may not be transgenic; examples of these traits include by are not limited to, male sterility, waxy starch, herbicide resistance, resistance for bacterial, fungal, or viral disease, insect resistance, male fertility, enhanced nutritional quality (such as higher protein content and oil content), industrial usage, yield stability and yield enhancement. These genes are generally inherited through the nucleus.

Further reproduction of the cultivar can occur by tissue culture and regeneration. Tissue culture of various plant tissues and regeneration of plants therefrom is well known and widely published. Thus, another aspect of one or more embodiments of the disclosure is to provide cells which, upon growth and differentiation, produce soybean plants having the physiological and morphological characteristics of a soybean cultivar selected from the group consisting of FTE 2009, FTE 3049, and FTE 3140.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, pods, leaves, stems, roots, root tips, anthers, and the like. Means for preparing and maintaining plant tissue culture are well known in the art. By way of example, a tissue culture comprising organs has been used to produce regenerated plants.

One or more embodiments of the disclosure also is directed to process for producing a soybean plant by crossing a first parent soybean plant with a second parent filed pea plant wherein the first or second parent soybean plant is a soybean plant of soybean cultivars FTE 2009, FTE 3049, or FTE 3140. Further, both first and second parent soybean plants can come from soybean cultivars FTE 2009, FTE 3049, or FTE 3140. Thus, any such process using the soybean cultivars FTE 2009, FTE 3049, or FTE 3140 are part of one or more embodiments of the disclosure: selfing, backcrosses, hybrid production, crosses to populations, and the like. All plants produced using soybean cultivars FTE 2009, FTE 3049, or FTE 3140 as at least one parent are within the scope of one or more embodiments of the disclosure, including those developed from varieties derived from soybean cultivars FTE 2009, FTE 3049, or FTE 3140. This soybean cultivar could be used in crosses with other, different, soybean plants to produce first generation (F₁) soybean hybrid seeds and plants with superior characteristics. The cultivar of one or more embodiments of the disclosure can also be used for transformation where exogenous genes are introduced and expressed by the cultivar of the one or more embodiments of the disclosure. Genetic variants created either through traditional breeding processes using soybean cultivars FTE 2009, FTE 3049, or FTE 3140 or through transformation of soybean cultivars FTE 2009, FTE 3049, or FTE 3140 by any number of protocols known to those of skill in the art are intended to be within the scope of one or more embodiments of the disclosure.

The seed of soybean cultivars FTE 2009, FTE 3049, and FTE 3140, the plant produced from the seed, the hybrid soybean plant produced from the crossing of the cultivar with any other soybean plant, hybrid seed, and various parts of the hybrid soybean plant can be utilized as a commercial commodity, or to make a commercial commodity, as is or in the production of a human food, livestock food, or new material in industry.

Soybeans can be used as a source of food due to its high protein and oil content. The high protein content makes it optimal for livestock feed. Human consumption of soy protein is growing in popularity as environmental studies show that vegetable protein is more sustainable than animal protein, while also providing human health benefits. Soybeans are an excellent source of protein for food products desired by industry and the general population.

Some varieties of soybean are used to make specialty foods for human consumption. Some varieties of soybean can be used to make tofu, soymilk, or extract protein for food products. Other varieties of soybean can be fermented to make foods such as miso paste and natto.

Production of edible protein ingredients from soybean offers a healthy, less expensive replacement for animal protein in meat-type a as well as in dairy-type products.

TABLE 2

| Variety | BU/A | BU/A | Variety | BU/A | BU/A |
|---|---|---|---|---|---|
| MG | MGI | MGI | MG | MGI | MGI |
| Replicate | 1 | 2 | Replicate | 1 | 2 |
| FTE 3049 | 64.1 | 61.6 | FTE 2009 | 65.3 | 67.1 |
| FTE 3140 | 64.1 | 61.6 | FTE 2034 | 51.6 | 56.8 |
| FTE 3008 | 53.1 | 60.0 | FTE 2252 | 53.1 | 58.8 |
| FTE 3009 | 55.5 | 57.7 | FTE 2253 | 53.1 | 60.0 |
| FTE 3015 | 55.5 | 59.9 | MN1011 | 56.9 | 58.34 |
| P92M10 | 59.4 | 61.8 | P91M10 | 59.8 | 62.4 |
| FTE 3151 | 53.1 | 60.0 | | | |

In Table 2, yield data of FTE 2009, FTE 3049, and FTE 3140 are shown contrasted to several competing varieties of commercial soybeans of similar maturity group. FTE 3049 and FTE 3140 and similar maturing varieties were grown in Minnesota. Columns 1 and 4 show the name of varieties tested. Columns 2, 3, 5, and 6 show the yield in bushels/acre (BU/A) for the disclosures and competing varieties.

The yield, protein, and oil results in Tables 2 & 3 were the result (for each replicate) of planting 300 seeds along a 16-foot row plot in maturity group 1 subgroup 4 area environmental conditions.

The trials were set up in a randomized block design, two replicates per variety, rows were 16 feet in length and 1 row was harvested. Tables 2 and 3 include commercially available varieties P91M10 and P92M10 (available through Pioneer), which are non-GMO varieties considered to give a high yield. Tables 2 and 3 include commercially available variety Minnesota MN1011, which is a non-GMO variety that is considered to have a resistance to Cyst Nematodes.

Major improvements in soybeans have been made by breeding varieties that are high in seeds per acre yield and high in protein and oil content (numbers are % protein and oil, “as is basis”) as measured by Near Infrared Spectroscopy.

TABLE 3 (A)
FTE 2009 & Comparison Cultivars: Protein and Oil Content

| Variety | Protein | Oil |
|---|---|---|
| FTE 2009 | 37.1 | 17.5 |
| FTE 2253 | 35.7 | 17.1 |
| FTE 2252 | 35.2 | 17.8 |
| FTE 2034 | 35.6 | 16.4 |
| MN1011 | 35.9 | 18.1 |
| P91M10 | 35.3 | 19.5 |

TABLE 3 (B)
FTE 3049 & Comparison Cultivars: Protein and Oil Content

| Variety | Protein | Oil |
|---|---|---|
| FTE 3049 | 37.8 | 18.3 |
| FTE 3008 | 35.0 | 18.1 |
| FTE 3009 | 36.7 | 17.9 |
| FTE 3015 | 35.1 | 16.7 |
| P92M10 | 36.5 | 18.7 |

TABLE 3 (C)
FTE 3140 & Comparison Cultivars: Protein and Oil Content

| Variety | Protein | Oil |
|---|---|---|
| FTE 3140 | 37.9 | 18.7 |
| FTE 3151 | 37.0 | 18.5 |
| FTE 3009 | 36.4 | 19.0 |
| FTE 3015 | 35.7 | 18.8 |
| 92M10 | 36.5 | 18.7 |

Tables 3A-3C comprise the protein and oil content in the cultivars of one or more embodiments of the disclosure and also in comparison cultivars. As the cultivar seeds of one or more embodiments of the disclosure are to be used for a variety of food products, high protein and oil content are important considerations for farmers choosing which cultivar they will plant. The seeds used for this evaluation of protein and oil content were collected from the plants grown for Table 2.

TABLE 4

| | Disease Test Results | | | | |
|---|---|---|---|---|---|
| Cultivar | IDC | SCN | PRR | WMR | SDS |
| FTE 2009 | Semi-Tot | Mod-Res | Semi-Tot | Tot | Res |
| FTE 3049 | Semi-Tot | Mod-Res | Semi-Tot | Tot | Res |
| FTE 3140 | Semi-Tot | Mod-Res | Semi-Tot | Tot | Res |
| FTE 2253 | Semi-Sus | — | Semi-Tol | Semi-Tol | Semi-Sus |
| FTE 2252 | Semi-Sus | — | Semi-Sus | Semi-Tol | Semi-Sus |
| FTE 2034 | Semi-Sus | — | Semi-Sus | Semi-Tol | Semi-Res |
| FTE 3008 | Sus | — | Semi-Sus | Semi-Sus | Semi-Sus |
| FTE 3009 | Semi-Sus | — | Semi-Tol | Semi-Sus | Semi-Sus |
| FTE 3015 | Semi-Sus | — | Semi-Sus | Semi-Tol | Semi-Sus |
| FTE 3151 | Semi-Sus | — | Semi-Sus | Semi-Tol | Semi-Res |

In Table 4, the results of testing for several soybean diseases are shown where susceptibility (Sus), resistance (Res), or tolerance (Tol) is reported for FTE 2009, FTE 3049, and FTE 3140 and other competing cultivars grown under the same environmental (maturity I) and time conditions

The cultivars of the current one or more embodiments of the disclosure (FTE 2009, FTE 3049, and FTE 3140) and several other cultivars commercially available from the inventor were evaluated for their susceptibility to iron deficiency chlorosis (IDC): Iron chlorosis deficiency is a challenge for soybean production. The symptoms of IDC include distinctive yellow leaves with dark green veins as well as stunted growth. When severe, the leaves turn yellow or white and the outer edges turn brown as the plant cells die. The symptoms can be seen spotted across fields as the soil changes. Soils with high pH and soils with soluble salts can reduce the availability of iron for the plant. Most soils contain an abundance of iron, but its availability can be reduced by the pH and salts in the soil. Susceptibility to IDC can be controlled genetically. When cultivars are tested for IDC they be considered tolerant (Tol): where no to minimal symptoms are observed, semi-tolerant (Semi-Tol): where symptoms are observed but there is no or minimal loss to yield and quality, semi-susceptible (Semi-Sus): symptoms are observed and there is some loss of yield and quality, and susceptible (Sus): symptoms are present and there is major loss of yield and quality.

Soybean cyst nematode (SCN), Heterodera glycines, is a roundworm that attacks the roots of a soybean plant and is found in most soybean growing areas around the world. SCN is a destructive pathogen that causes 1.5 billion dollars of US soybean losses annually and is the number one cause of soybean loss. In central and northern Minnesota, the cyst nematode can complete three, or even four, life cycles in a single growing season and can be prohibitively expensive to eradicate with chemicals or other management practices. For this reason, genetic SCN resistance is usually the source of prevention for farmers.

Soybean cyst nematode (SCN) screening (testing) was performed on cultivars FTE 2009, FTE 3049, and FTE 3140 at the plant pathology lab at Iowa State University. Results were given as follows: Resistant (Res): 0-14 cysts present; Moderately Resistant (Mod-Res): 15-42 cysts present; Moderately Susceptible (Mod-Sus): 43-85 cysts present; Susceptible (Sus): 86 or more cysts present.

Phytophthora root rot (Phytophthora sojae) is of economic importance in fields with poor drainage, or areas with low-lying areas that are prone to flooding. Symptoms of Phytophthora root rot include brown lesions, which can grow high on a soybean stem and girdle the stem, causing stunting or death of the plant. Phytophthora sojae can infect soybean plants at any stage of their growth. Varieties of soybean that are not fully susceptible could be stunted in growth, but not killed. Phytophthora root rot is best managed by choosing cultivar with some resistance.

Several cultivars of the current one or more embodiments of the disclosure (including FTE 2009, FTE 3049, and FTE 3140) and several commercially available cultivars of the inventor were evaluated as to their tolerance or susceptibility to Phytophthora root rot. The results were based on the following ratings:

Tolerant (Tol): No dark brown lesions on the stem; no yellowing or wilting of plant tissue. No stunting of plant growth, Semi Tolerant (Semi-Tol): Some yellowing of leaf tissue; small brown lesions observed on the lower portion of the plant reaching the 1^{st} of second node, Semi Susceptible (Semi-Sus): Yellowing and wilting of plant tissue; brown lesions observed from the lower portion of the plant to several nodes high, Susceptible (Sus): Plants are completely wilted and dead

White mold (Sclerotinia stem rot, Sclerotinia sclerotiorum) is an often lethal fungal disease of soybeans in North America. White mold occurs most commonly in environments where soybeans comprise dense canopies in cool and moist environments. White mold causes wilting of leaves and plant death, as well as bleaching at the base of the stem. Genetic tolerance to white mold is available.

Several cultivars of the current one or more embodiments of the disclosure (including FTE 2009, FTE 3049, and FTE 3140) and several commercially available cultivars of the inventor were evaluated as to their tolerance or susceptibility to white mold and were scored in a similar way as described above in IDC.

Sudden Death Syndrome (SDS) (Fusarium virguliforme) is a disease that causes the second highest damage in the United States after SCN. Diseased plants comprise rotted taproots and lateral roots resulting in a high plant death rate late in the season. Foliar symptoms start to appear after flowering. Leaves of infected plants initially show scattered yellow spots between leaf veins. There is genetic resistance to SDS.

Several cultivars of the current one or more embodiments of the disclosure (including FTE 2009, FTE 3049, and FTE 3140) and several commercially available cultivars of the inventor were evaluated for their resistance or susceptibility to Sudden Death Syndrome (Fusarium virguliforme).

In Table 4, Row 2 lists the abbreviated names for all plant diseases tested. Column 1 lists the cultivar names that were tested for the described plant diseases. Columns 2-6 show the susceptibility, tolerance, or resistance results of the pathology tests for all listed cultivars.

Deposit Information

A deposit of the proprietary soybean cultivars designated FTE 2009, FTE 3049, and FTE 3140 disclosed above and recited in the appended claims has been made with the American Type Culture Collection (ATCC), 10801 University Boulevard, Manassas, Va. 20110. The date of deposit was Apr. 4, 2018. The deposits of 2500 seeds each was taken from the same deposit maintained by FTE, since prior to the filing date of this application. All restrictions upon the deposit have been removed, and the deposit is intended to meet all of the requirements of 37 CFR § 1.801-1.809. The ATCC accession numbers are PTA-125044, PTA-125045, and PTA-125046. The deposits will be maintained in the depository for a period of 30 years, or 5 years after the last request, or for the effective life of the patent, whichever is longer, and will be replaced as necessary during that period.

In sum, it is important to recognize that this disclosure has been written as a thorough teaching rather than as a narrow dictate or disclaimer. Reference throughout this specification to “one embodiment”, “an embodiment”, or “a specific embodiment” means that a particular feature, structure, or characteristic described in connection with the embodiment is included in at least one embodiment and not necessarily in all embodiments. Thus, respective appearances of the phrases “in one embodiment”, “in an embodiment”, or “in a specific embodiment” in various places throughout this specification are not necessarily referring to the same embodiment. Furthermore, the particular features, structures, or characteristics of any specific embodiment may be combined in any suitable manner with one or more other embodiments. It is to be understood that other variations and modifications of the embodiments described and illustrated herein are possible in light of the teachings herein and are to be considered as part of the spirit and scope of the present subject matter.

The term “or” as used herein is generally intended to mean “and/or” unless otherwise indicated. Combinations of components or steps will also be considered as being noted, where terminology is foreseen as rendering the ability to separate or combine is unclear.

As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise. Variation from amounts specified in this teaching can be “about” or “substantially,” so as to accommodate tolerance for such as acceptable manufacturing tolerances.

The foregoing description of illustrated embodiments, including what is described in the Abstract and the Summary, and all disclosure and the implicated industrial applicability, are not intended to be exhaustive or to limit the subject matter to the precise forms disclosed herein. While specific embodiments of, and examples for, the subject matter are described herein for teaching-by-illustration purposes only, various equivalent modifications are possible within the spirit and scope of the present subject matter, as those skilled in the relevant art will recognize and appreciate. As indicated, these modifications may be made in light of the foregoing description of illustrated embodiments and are to be included, again, within the true spirit and scope of the subject matter disclosed herein.

Although the foregoing one or more embodiments of the disclosure has been described in some detail by way of illustration and example for purposes of clarity and understanding, it will be clear that certain changes and modifications such as single gene modifications and mutations, somaclonal variants, variant individuals selected from large populations of the parts of the instant soybean cultivar and the like may be practiced within the scope of the one or more embodiments, as limited only by the scope of claims.

1. A seed of one of a group of soybean cultivars consisting of FTE 2009, FTE 3049, and FTE 3140, wherein a representative sample of seed of said cultivar was deposited under ATCC Accession No. PTA-124920.

2. A soybean plant, or a part thereof, produced by growing the seed of claim 1.

3. A tissue culture of regenerable cells produced from the plant of claim 2, wherein said cells of the tissue culture are produced from a plant part selected from the group comprising leaf, pollen, embryo, cotyledon, hypocotyl, meristematic cell, root, root tip, pistil, anther, flower, stem, and pod.

4. A protoplast produced from the plant of claim 2.

5. A protoplast produced from the tissue culture of claim 3.

6. (canceled)

7. A soybean plant, or part thereof, of claim 2, wherein the plant comprises a disease resistance to iron deficiency chlorosis, cyst nematode, Phytophthora root rot, sudden death syndrome, white mold, and combinations thereof.

8. A soybean plant, or part thereof, of claim 2, wherein the plant produces a seed with a seed weight of about 18 to 22 g/100 seeds.

9. A soybean plant, or part thereof, of claim 2, wherein the yield is greater than 60 bushels of seed per acre.

10. The seed of claim 1, wherein the seeds contain greater than about 35% protein.

11. The seed of claim 1, wherein the seeds contain greater than about 17% oil.

12. A process for producing an F₁ hybrid soybean seed, wherein the process comprises crossing the plant of claim 2 with a different soybean plant, and harvesting the resultant F₁ hybrid soybean seed.

13. (canceled)

14. A hybrid soybean plant, or a part thereof, produced by growing said hybrid seed of claim 12.

15. A process of producing a disease resistant soybean plant wherein the process comprises transforming a soybean plant with a transgene from the plant, or part of the plant, of claim 2 that confers disease resistance; wherein the disease is selected from the group comprising iron deficiency chlorosis, cyst nematode, Phytophthora root rot, sudden death syndrome, white mold, and combinations thereof.

16. (canceled)

17. The soybean plant, or a part thereof, of claim 12, wherein the plant or plant part is resistant to a disease selected from the group comprising iron deficiency chlorosis, cyst nematode, Phytophthora root rot, sudden death syndrome, white mold, and combinations thereof.

18.-19. (canceled)

20. A process of producing a soybean plant that produces seeds with properties selected from the group consisting of yield greater than 60 bushels per acre, seed protein content of greater than about 35%, seed oil content of greater than 17%, and seed weight less than about 22 g/100 seeds; wherein the process comprises genetic engineering or natural breeding.

21. (canceled)

22. A process of introducing a desired trait into a soybean cultivar selected from the group of FTE 2009, FTE 3049, and FTE 3140 of claim 1, wherein the process involves genetic engineering or natural breeding techniques.

23. (canceled)

24. The plant of claim 14, wherein the plant has a resistance to at least one disease selected from the group of iron chlorosis, cyst nematode, Phytophthora root rot, sudden death syndrome, white mold, and combinations thereof.

25.-35. (canceled)

36. A process of producing a commodity plant product comprising:
obtaining the soybean plant of claim 2 or a part thereof; and
producing the commodity plant product therefrom.

37. The process of claim 36, wherein the commodity plant product is protein powder, protein concentrate, protein isolate, soybean fiber, soybean starch, soybean meal, soybean flour, soybean hulls, soybean oil, and combinations thereof.

38.-40. (canceled)

41. The commodity plant product of claim 36, wherein the commodity plant product is further modified by treatments selected from the group of heating, milling, cooking, extruding, steaming, hydrolyzing, emulsifying, hydrogenating, acidifying, buffering, chemical modifying, and combinations thereof.

42. The food product of claim 40, wherein the food product is further modified by treatments selected from the group of heating, milling, cooking, extruding, steaming, hydrolyzing, emulsifying, hydrogenating, acidifying, buffering, chemical modifying, and combinations thereof.
